(19) United States
(12) Patent Application Publication
(10) Pub. No.: US 2011/0060238 A1
(43) Pub. Date: Mar. 10, 2011

(54) Systems and methods for intra-operative physiological functional stimulation

(75) Inventors: MICHAEL R. HAUSMAN, NEW YORK, NY (US); GEOFFREY B. THROPE, SHAKER HEIGHTS, OH (US); ROBERT B. STROTHER, WILLOUGHBY HILLS, OH (US); JOSEPH J. MRVA, EUCLID, OH (US)

(73) Assignee: Checkpoint Surgical, LLC

(21) Appl. No.: 12/806,691

(22) Filed: Aug. 19, 2010

Related U.S. Application Data
(63) Continuation-in-part of application No. 11/651,165, filed on Jan. 9, 2007.
Application No. 11/651,165 is a continuation-in-part of application No. 11/099,848, filed on Apr. 6, 2005.
(60) Provisional application No. 60/657,277, filed on Mar. 1, 2005.
(60) Provisional application No. 61/338,312, filed on Feb. 16, 2010.

(51) Int. Cl. A61B 5/04

(52) U.S. Cl. 600/544

(57) ABSTRACT

Improved assemblies, systems, and methods provide safeguarding against tissue injury during surgical procedures and/or identify nerve damage occurring prior to surgery and/or verify range of motion or attributes of muscle contraction during reconstructive surgery. Embodiments of methods according to the present invention provide the ability to intra-operatively simulate post-operative physiologic function of a body part. Such methods may be used during various surgical procedures, including tendon transfer procedures. Included are one or more steps of confirming paralysis or a weakened condition of a body part, confirming responsivity or operability of transfer tissue to supplement the functionality of the paralyzed or weakened part, and intra-operatively simulating post-operative functionality of the transfer tissue to enhance and/or predict the outcome of the surgical procedure.

### RELATED APPLICATIONS

This application is a continuation-in-part of co-pending U.S. patent application Ser. No. 11/651,165, filed Jan. 9, 2007, and entitled “Systems and Methods for Intra-Operative Stimulation,” which is a continuation-in-part of U.S. patent application Ser. No. 11/099,848, filed Apr. 6, 2005, and entitled “Systems and Methods for Intra-Operative Stimulation,” which claims the benefit of U.S. Provisional Patent Application Ser. No. 60/657,277, filed Mar. 1, 2005, and entitled “Systems and Methods for Intra-Operative Stimulation,” each of which is incorporated herein by reference in its entirety.

This application also claims the benefit of U.S. Patent Application Ser. No. 61/338,312, filed Feb. 16, 2010, and entitled “Systems and Methods for Intra-Operative Stimulation,” which is incorporated herein by reference in its entirety.

### FIELD OF THE INVENTION

The invention relates generally to tissue identification and integrity testing, and more particularly to systems and methods for safeguarding against nerve and muscle injury during surgical procedures, location and stimulation of nerves and muscles, identification and assessment of nerve and muscle integrity following traumatic injuries, and verification of range of motion and attributes of muscle contraction during reconstructive surgery.

### BACKGROUND OF THE INVENTION

Even with today's sophisticated medical devices, surgical procedures are not risk-free. Each patient's anatomy differs, requiring the surgeon to be ever vigilant to these differences so that the intended result is accomplished. The positioning of nerves and other tissues within a human or animal's body is one example of how internal anatomy differs from patient to patient. While these differences may be slight, if the surgeon fails to properly identify one or several nerves, the nerves may be bruised, stretched, or even severed during an operation. The negative effects of nerve damage can range from lack of feeling on that part of the body to loss of muscle control.

Traumatic injuries often require surgical repair. Determining the extent of muscle and nerve injury is not always possible using visual inspection. Use of an intra-operative stimulator enables accurate evaluation of the neuromuscular system in that area. This evaluation provides valuable knowledge to guide repair/reconstructive surgery following traumatic injury, and when performing a wide range of surgeries.

### SUMMARY OF THE INVENTION

The invention provides devices, systems, and methods for intra-operative stimulation. The intra-operative stimulation enables accurate evaluation of the neuromuscular system to guide repair or reconstructive surgery.

In one embodiment of a method according to the present invention, a method of physiologic functional simulation may be conducted during a surgical procedure affecting functional change of muscle tissue innervated by working nerve tissue. An example of such surgical procedure may be a tendon transfer procedure. The method may include the step of relocating first tendon tissue to an unnatural position. In a first application step, a first electrical stimulation may be applied to first muscle tissue into which the first tendon tissue is naturally inserted. In a first observation step, a neural response may be observed in response to the first electrical stimulation. While the relocating step may be performed either before or after the first application step, it is preferably performed before the first application step.

According to one aspect of the present invention, the first tendon tissue is a first tendon having two ends, wherein a first end is naturally inserted into the first muscle tissue and a second end is coupled to a first bone. The method may further include the step of incising all, such as severing, or part of the first tendon at or near the first bone to form a tendon free end. The tendon free end may be coupled to a second body part, which may be a second bone, after which the tendon may span a joint disposed between the second bone and a third bone.

According to another aspect of a method according to the present invention, the neural response observed may be a movement of the second body part through a predetermined range of motion. An observed neural response may also or additionally be a twitch of the first muscle.

According to still another aspect of a method according to the present invention, the tendon free end may be coupled to the second body part by stitching the first tendon to one of a second tendon, a second muscle, and a periosteum of the second bone.

Another embodiment of a method of physiologic functional simulation according to the present invention further includes the steps of providing a device to be used in the first application step. The device may include a housing extending along a housing longitudinal axis between a housing proximal end and a housing distal end. Electrical stimulation generation circuitry may be completely, or at least substantially, contained within the housing. An operative element may extend from the housing proximal end, the operative element including an electrode operatively coupled to the electrical stimulation generation circuitry. Also disposed completely, or at least substantially within the housing, is a power supply that is electrically coupled to the electrical stimulation generation circuitry. The first application step may include the step of applying the first electrical stimulation from the electrode to the first muscle tissue.

According to an aspect of another embodiment according to the present invention, the provided device is completely or at least substantially sterile.

According to another aspect of a method according to the present invention, the provided device is capable of delivering, through the electrode sufficient electrical stimulation to activate the first muscle tissue when the electrode is in physical contact therewith.

According to yet another aspect of a method according to the present invention, a provided device may be carried by a single human hand. Such method may further include the step of manipulating, with the single human hand, the device to change an electrical stimulation parameter. The manipulating step may occur during the first application step.

According to another aspect of a provided device according to the present invention, the device may further include an electronic visual indicator operatively coupled to the stimulation circuitry. A method may further include a step of observing a first visual indication provided by the visual indicator, the first visual indication being indicative of electrical stimulation flowing at least partially through the targeted tissue region. The method may further include a step of observing a second visual indication provided by the visual indicator, the second visual indication being indicative of electrical power supplied to the stimulation circuitry by the power supply. The first visual indication may be an illumination of a first color and the second visual indication may be an illumination of a second color, the second color being different from the first color. The visual indicator may be an illumination device that is radially visible from 360 degrees around the longitudinal axis of the handle of the provided device.

In another embodiment of a method according to the present invention, a method of physiologic functional simulation may be conducted during a surgical procedure affecting functional change of muscle tissue innervated by working nerve tissue. An example of such surgical procedure may be a tendon transfer procedure. The method may include the step of relocating first tendon tissue to an unnatural position. In a first application step, a first electrical stimulation may be applied to first muscle tissue into which the first tendon tissue is naturally inserted. In a first observation step, a neural response may be observed in response to the first electrical stimulation. The first application step and the first observation step may be performed before the relocating step to, e.g., determine responsivity of viable muscle and/or nerves prior to the transfer. After the relocating step, in a second application step, a second electrical stimulation may be applied to the first muscle tissue. In a second observation step, a second neural response to the second electrical stimulation may be observed.

According to another aspect of a method according to the present invention, it may be desirable to confirm paralysis of a second muscle, the function of which will be supplemented by the first muscle tissue. This confirmation is preferably carried out prior to the relocating step. The confirming step may be performed, in a confirmation step, by applying a second electrical stimulation to the second muscle, the electrical stimulation having an amplitude of about twenty milliamps.

According to another aspect of a method according to the present invention, in addition to performing the confirmation step, the first application step and the first observation step prior to the relocating step, it may be desirable to, after the relocating step, apply a third electrical stimulation to the first muscle tissue. A second neural response may be observed in response to the third electrical stimulation. In this way, in addition to verifying paralysis of a damaged body part and the working order of a body part to be transferred prior to relocating the transfer tissue, the post-operative functionality of the transfer tissue may be simulated.

Features and advantages of the inventions are set forth in the following Description and Drawings, as well as the appended description of technical features.

### BRIEF DESCRIPTION OF THE DRAWINGS

FIG. 1 is a diagrammatic view of a system usable in association with a family of different monitoring and treatment devices for use in different medical procedures.

FIG. 2 is a perspective view showing an exemplary embodiment of the system shown in FIG. 1, the stimulation control device being removably coupled to a stimulation probe, and showing the stimulation signal path through the system.

FIG. 3A is a side view with a portion broken away and in section showing the stimulation probe having the stimulation control device embedded within the stimulation probe.

FIG. 3B is a side view with a portion broken away and in section showing the stimulation probe having the stimulation control device embedded within the stimulation probe, and showing an optional needle-like return electrode.

FIG. 3C is a side view with a portion broken away and in section showing an additional embodiment of the stimulation probe having a housing that includes a gripping base and a flexible nose cone, and an illuminating ring indicator.

FIG. 4A is a side view of the stimulation probe of FIG. 3c, showing the users hand in a position on the stimulation probe to move the flexible nose cone.

FIG. 4B is a side view of the stimulation probe of FIG. 4A, showing the users hand flexing the flexible nose cone.

FIG. 5 is a side view with a portion broken away and in section showing elements of the flexible nose cone, the ring indicator, and the gripping base.

FIG. 6 is a graphical view of a desirable biphasic stimulus pulse output of the stimulation device.

FIG. 7 is a view showing how the geometry of the stimulation control device shown in FIG. 2 aids in its positioning during a surgical procedure.

FIG. 8 is a block diagram of a circuit that the stimulation control device shown throughout the Figs. can incorporate.

FIGS. 9A and 9B are perspective views showing the stimulation control device in use with a cutting device.

FIGS. 10A and 10B are perspective views showing the stimulation control device in use with a drilling or screwing device.

FIGS. 11A and 11B are perspective views showing the stimulation control device in use with a pilot auger device.

FIGS. 12A and 12B are perspective views showing the stimulation control device in use with a fixation device.

FIG. 13 is a plane view of a kit used in conjunction with the stimulation probe shown in FIG. 3C, and including the stimulation probe and instructions for use.

FIG. 14 is a perspective view of the stimulation probe shown in FIG. 3C.

FIG. 15 is an exploded view of the stimulation probe shown in FIG. 14.

FIG. 16 is a flowchart depicting steps included in an embodiment of a method of physiological functional simulation according to the present invention.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF PREFERRED EMBODIMENTS

This Specification discloses various systems and methods for safeguarding against nerve, muscle, and tendon injury during surgical procedures or confirming the identity and/or location of nerves, muscles, and tendons and evaluating their function or the function of muscles enervated by those nerves. The systems and methods are particularly well suited for assisting surgeons in identification of nerves and muscles in order to assure nerve and muscle integrity during medical procedures using medical devices such as stimulation monitors, cutting, drilling, and screwing devices, pilot augers, and fixation devices. For this reason, the systems and methods will be described in the context of these medical devices.

The systems and methods desirably allow the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Still, it should be appreciated that the disclosed systems and methods are applicable for use in a wide variety of medical procedures with a wide variety of medical devices. By way of non-limiting example, the various aspects of the invention have application in procedures requiring grasping medical devices and internal viewing devices as well.

### I. Overview of the System

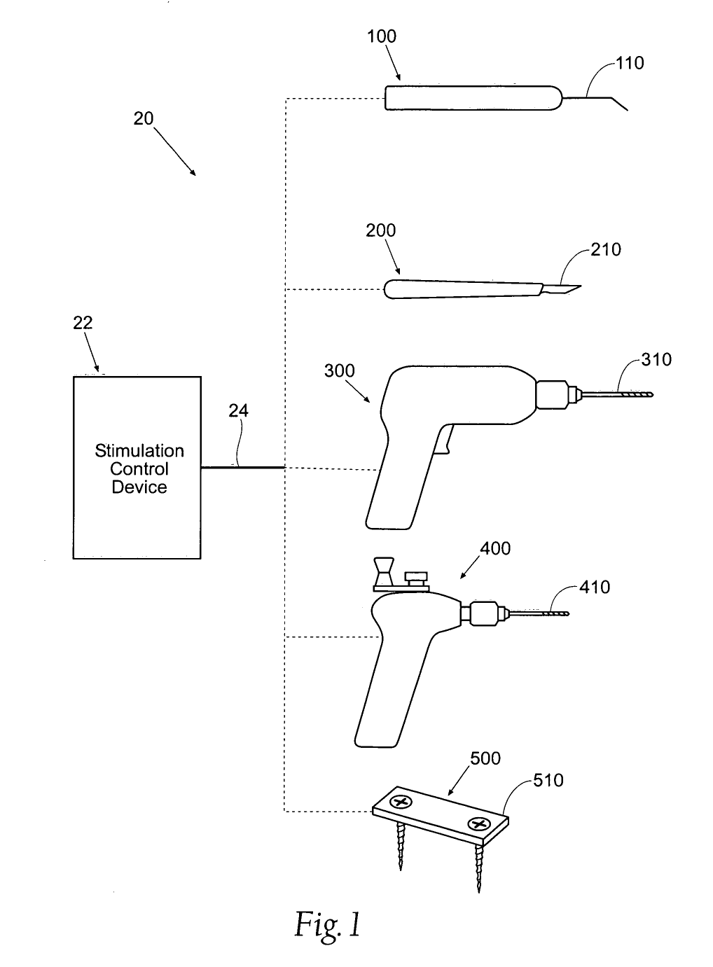

FIG. 1 shows an illustrative system 20 for locating and identifying tissue and safeguarding against tissue and/or bone injury during surgical procedures. In the illustrated embodiment, the system 20 is configured for locating, monitoring, and stimulating tissue and other structures throughout the body. The system 20 includes a stimulation control device 22 operating individually or in conjunction with one or more of a family of stimulating medical devices including, for example, a stimulation monitor or probe 100, a cutting device 200, a drilling or screwing device 300, a pilot auger 400, and a fixation device 500.

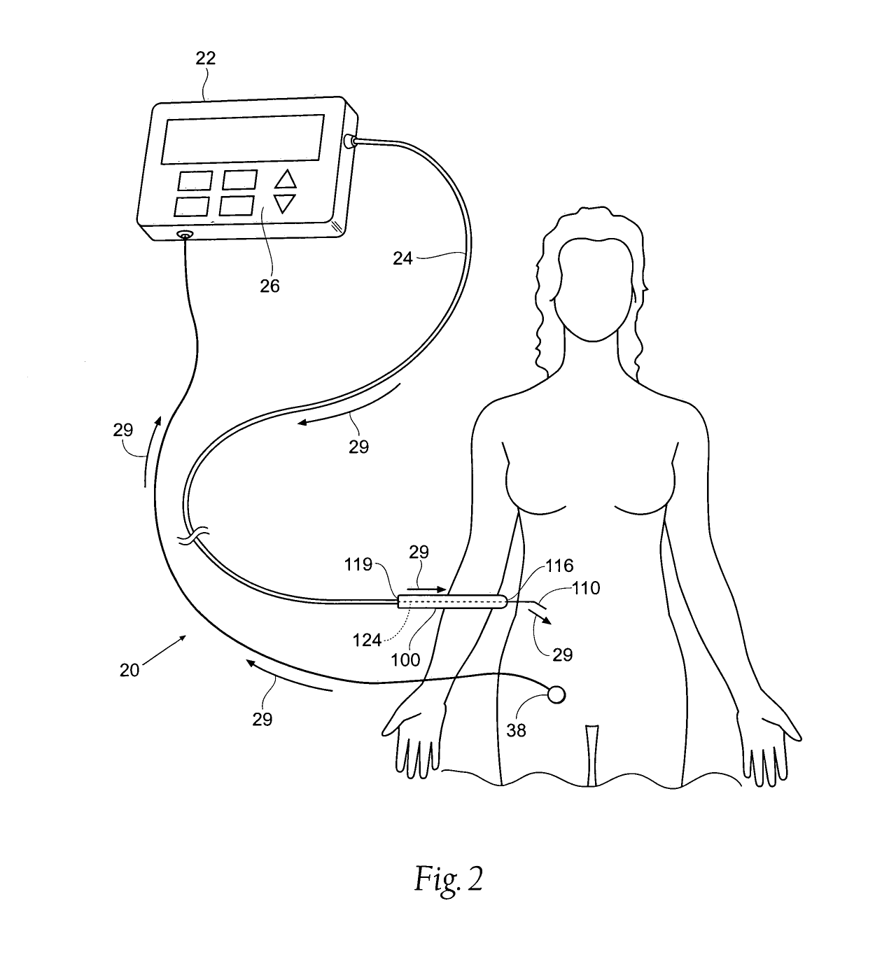

In an exemplary embodiment, and as can be seen in FIG. 2, the stimulation control device 22 functions in the system 20 to generate an electrical stimulation signal 29. The stimulation signal 29 flows from the stimulation control device 22 through a lead 24 to a medical device (e.g., stimulation probe 100). The stimulation signal 29 then flows through a predefined insulated path 124 within the stimulation probe 100 and to an operative element, such as an electrically conductive surface, i.e., a coupled electrode 110. The electrode 110 is to be positioned on or near a region of a patient to be stimulated. In monopolar operation, a return electrode (or indifferent electrode) 38 provides an electrical path from the body back to the control device 22. The stimulation control device 22 may operate in a monopolar or bipolar configuration, as will be described in greater detail later.

The stimulation signal 29 is adapted to provide an indication or status of the device. The indication may include a physical motor response (e.g., twitching), and/or one or more visual or audio signals from the stimulation control device 22, which indicate to the surgeon the status of the device, and/or close proximity of the electrode 110 to a nerve, or a muscle, or a nerve and a muscle. The stimulation control device may also indicate to the surgeon that the stimulation control device is operating properly and delivering a stimulus current.

### II. Medical Devices

The configuration of the stimulating medical devices that form a part of the system can vary in form and function. Various representative embodiments of illustrative medical devices will be described.

A. Stimulation Probe

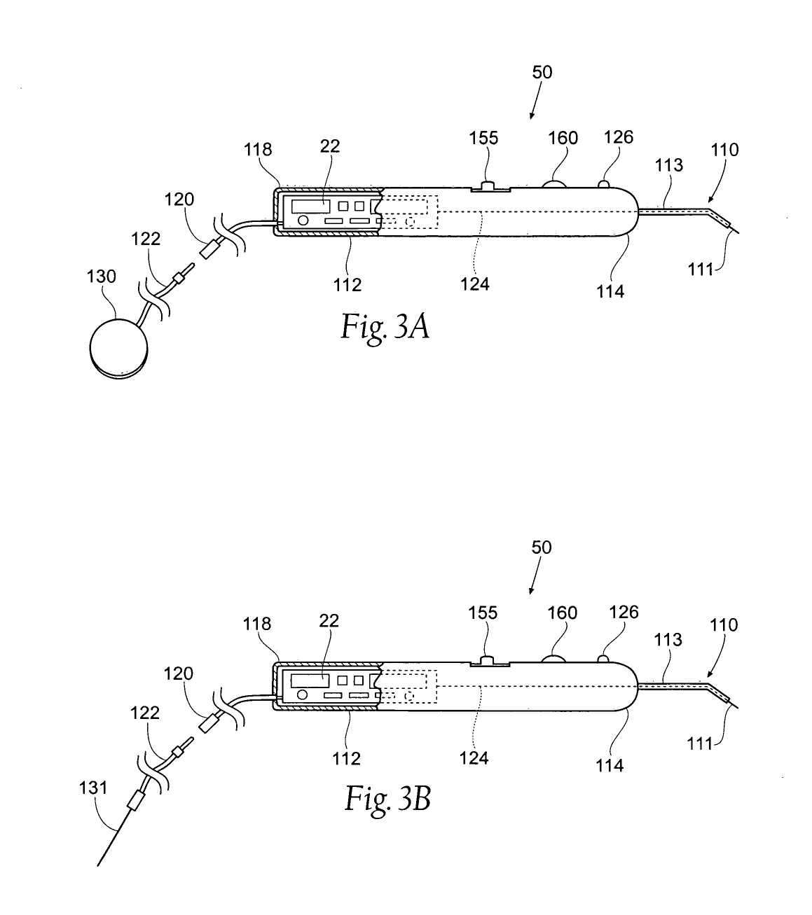

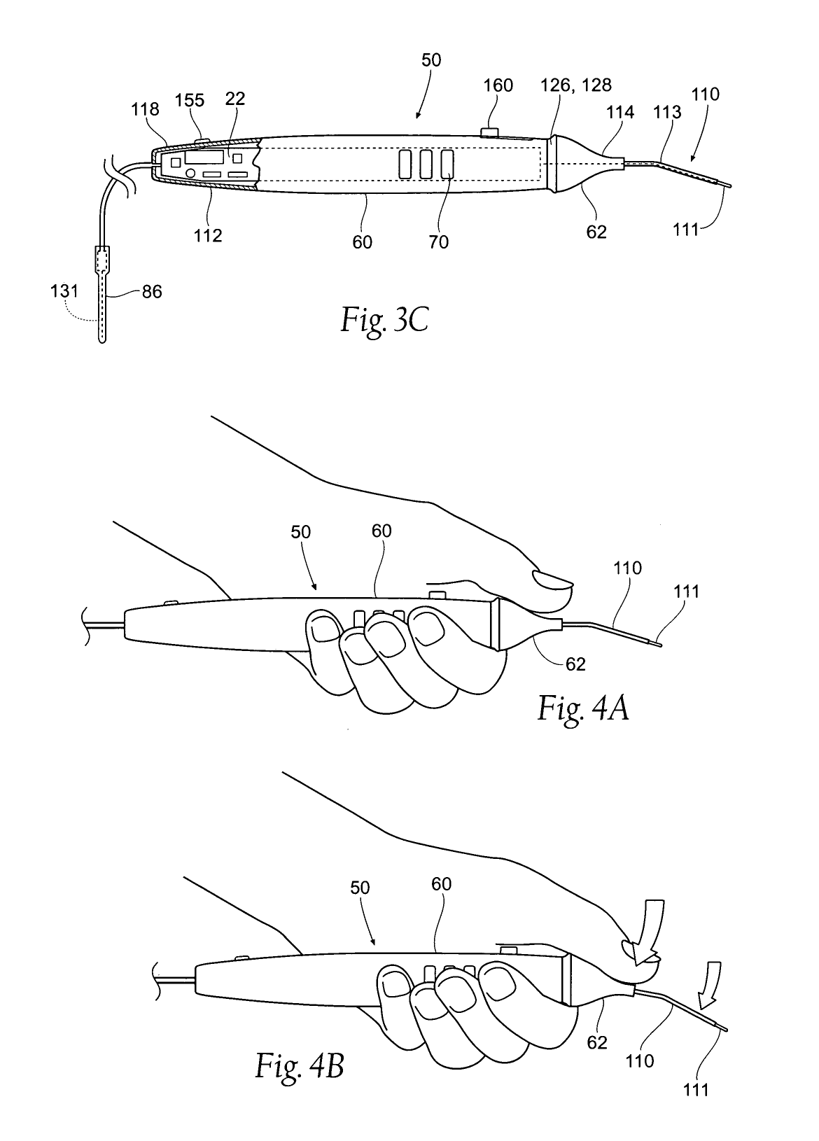

FIGS. 3A to 3C show various embodiments of a hand held stimulation monitor or probe 50 for identification and testing of nerves and/or muscles during surgical procedures. As shown, the stimulation probe 50 may accommodate within a generally tubularly housing 112 the electrical circuitry of a stimulation control device 22. The stimulation probe 50 is desirably an ergonomic, sterile, single use instrument intended for use during surgical procedures to identify nerves and muscles, muscle attachments, or to contract muscles to assess the quality of surgical interventions or the need for surgical interventions, or to evaluate the function of nerves already identified through visual means. The stimulation probe 50 may be sterilized using ethylene oxide, for example.

The stimulation probe 50 is preferably sized small enough to be held and used by one hand during surgical procedures, and is ergonomically designed for use in either the left or right hand. In a representative embodiment, the stimulation probe 50 may have a width of about 20 millimeters to about 30 millimeters, and desirably about 25 millimeters. The length of the stimulation probe 50 (not including the operative element 110) may be about 18 centimeters to about 22 centimeters, and desirably about 20 centimeters. The operative element 110 may also include an angle or bend to facilitate access to deep as well as superficial structures without the need for a large incision. The operative element 110 will be described in greater detail later. A visual or audio indicator 126 incorporated with the housing 112 provides reliable feedback to the surgeon as to the request and delivery of stimulus current.

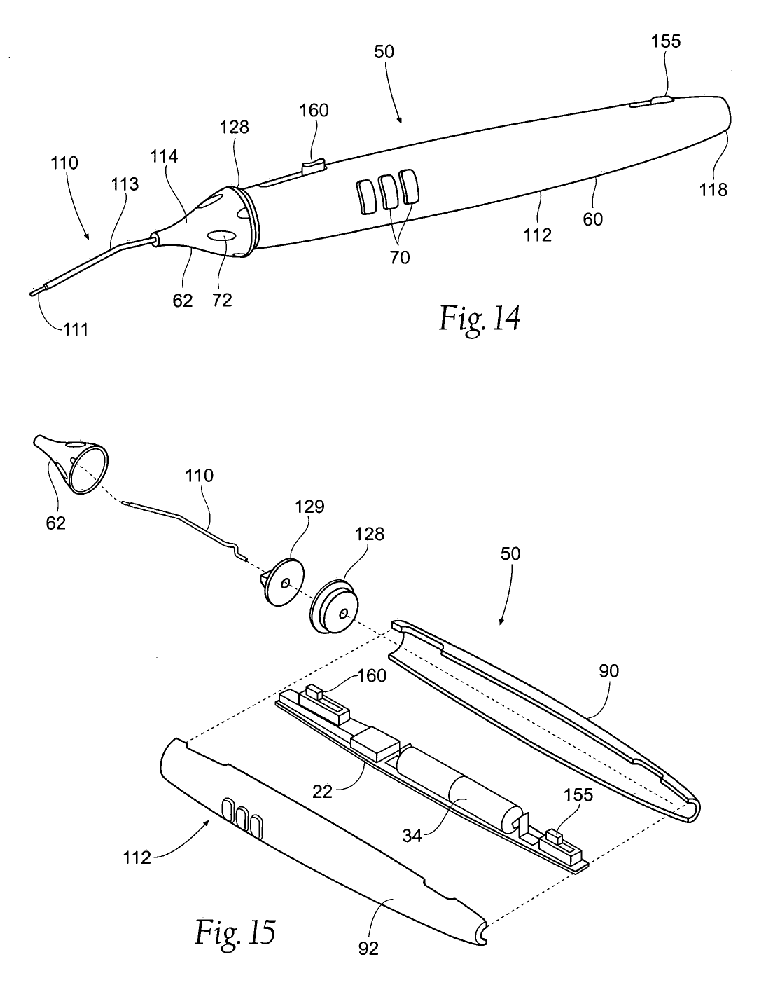

In one embodiment shown in FIGS. 3C and 14, the stimulation probe 50 includes a housing 112 that comprises a gripping base portion 60 and an operative element adjustment portion 62. The operative element 110 extends from the proximal end of the adjustment portion 62. In order to aid the surgeon in the placement of the operative element 110 at the targeted tissue region, the adjustment portion, as will be described as a nose cone 62, may be flexible. This flexibility allows the surgeon to use either a finger or a thumb positioned on the nose cone 62 to make fine adjustments to the position of stimulating tip 111 of the operative element 110 at the targeted tissue region (see FIGS. 4A and 4B). The surgeon is able to grasp the gripping base 60 with the fingers and palm of the hand, and position the thumb on the nose cone 62, and with pressure applied with the thumb, cause the stimulating tip 111 to move while maintaining a steady position of the gripping base portion 62. This flexible nose cone 62 feature allows precise control of the position of the stimulating tip 111 with only the movement of the surgeon's thumb (or finger, depending on how the stimulating probe is held).

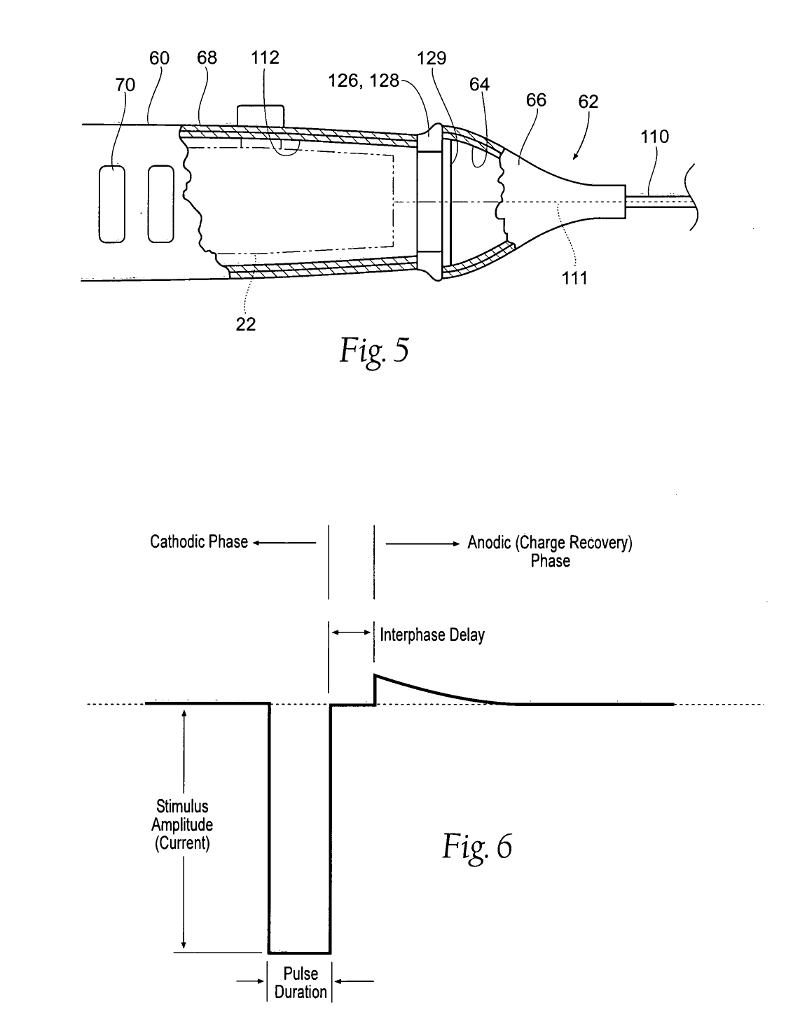

The flexible nose cone 62 may comprise a single element or it may comprise at least an inner portion 64 and an outer portion 66, as shown in FIG. 5. In order to facilitate some flexibility of the proximal portion 114 of the stimulation probe 50, the inner portion 64 of the nose cone 62 may be made of a thermoplastic material having some flexibility. One example may be LUSTRAN® ABS 348, or similar material. The outer portion 66 may comprise a softer over molded portion and may be made of a thermoplastic elastomer material having some flexibility. One example may be VERSAFLEX™ OM 3060-1 from GLS Corp. The nose cone 62 is desirably generally tapered. For example, the nose cone 62 may be rounded, as shown in FIGS. 3A and 3B, or the nose cone may be more conical in shape, as shown in FIG. 3C.

The nose cone 62 may also include one or more features, such as ribs or dimples 72, as shown in FIG. 14, to improve the gripping, control, and stability of the stimulation probe 50 within the surgeon's hand.

The gripping base portion 60 of the housing 112 may also include an overmolded portion 68. The overmolded portion 68 may comprise the full length of the gripping base portion 60, or only a portion of the gripping base 60. The soft overmolded portion 68 may include one or more features, such as dimples or ribs 70, as shown, to improve the gripping, control, and stability of the stimulation probe 50 within the surgeon's hand. The overmolded portion 68 may comprise the same or similar material as the thermoplastic elastomer material used for the outer portion 66 of the flexible nose cone 62.

In one embodiment, the stimulation probe 50 includes a housing 112 that carries an insulated lead 124. The insulated lead 124 connects the operative element 110 positioned at the housing's proximal end 114 to the circuitry 22 within the housing 112 (see FIG. 3A). It is to be appreciated that the insulated lead is not necessary and the operative element 110 may be coupled to the circuitry 22 (see FIG. 3C). The lead 124 within the housing 112 is insulated from the housing 112 using common insulating means (e.g., wire insulation, washers, gaskets, spacers, bushings, and the like). The conductive tip 111 of the operative element 110 is positioned in electrical conductive contact with at least one muscle, or at least one nerve, or at least one muscle and nerve.

As shown, the stimulation probe 50 is mono-polar and is equipped with a single operative element (i.e., electrode) 110 at the housing proximal end 114. A return electrode 130, 131 may be coupled to the stimulation probe 50 and may be any of a variety of electrode types (e.g., paddle, needle, wire, or surface), depending on the surgical procedure being performed. As shown, the various return electrodes 130, 131 are coupled to the housing distal end 118. In an alternative embodiment, the stimulation device 50 itself may be bipolar by including a return electrode in the operative element 110, which precludes the use of a return electrode coupled to the stimulation probe 50.

As shown and described, the stimulation probe 50 may accommodate within the housing 112 the electrical circuitry of a stimulation control device 22. In this arrangement, the stimulation probe 50 may have one or more user operable controls. Two are shown—155 and 160. Power switch 155 serves a dual purpose of turning the stimulation probe 500N and OFF (or standby), and also can be stepped to control the stimulation signal amplitude selection within a predefined range (e.g., 0.5, 2.0, and 20 mA). In this configuration, the switch may be a four position switch. Before the first use of the stimulation probe 50, the power switch 155 is in the OFF position and keeps the stimulation probe off. After the stimulation probe 50 has been turned ON—by moving the switch 155 to an amplitude selection—the OFF position now corresponds to a standby condition, where no stimulation would be delivered. In one embodiment, once the stimulation probe 50 has been turned on, it cannot be turned off, it can only be returned to the standby condition and will remain operational for a predetermined time, e.g., at least about seven hours. This feature is intended to allow the stimulation probe 50 to only be a single use device, so it can not be turned OFF and then used again at a later date.

The pulse control device 160 allows for adjustment of the stimulation signal pulse width from a predefined range (e.g., about zero to about 200 microseconds). In one embodiment, the pulse control 160 may be a potentiometer to allow a slide control to increase or decrease the stimulation signal pulse width within the predefined range.

The stimulation pulse may have a non-adjustable frequency in the range of about 10 Hz to about 20 Hz, and desirably about 16 Hz.

As a representative example, the stimulation pulse desirably has a biphasic waveform with controlled current during the cathodic (leading) phase, and net DC current less than 10 microamps, switch adjustable from about 0.5 milliamps to about 20 milliamps, and pulse durations adjustable from about zero microseconds up to about 200 microseconds. A typical, biphasic stimulus pulse is shown in FIG. 6.

The operative element 110 exits the housing 112 at the proximal end 114 to deliver stimulus current to the excitable tissue. The operative element 110 comprises a length and a diameter of a conductive material, and is desirably fully insulated with the exception of the most proximal end, e.g. about 1.0 millimeters to about 10 millimeters, and desirably about 4 millimeters to about 6 millimeters, which is non-insulated and serves as the stimulating tip or surface (or also referred to as active electrode) 111 to allow the surgeon to deliver the stimulus current only to the intended tissue. The small area of the stimulating surface 111 (the active electrode) of the operative element 110 ensures a high current density that will stimulate nearby excitable tissue. The insulation material 113 may comprise a medical grade heat shrink.

The conductive material of the operative element 110 comprises a diameter having a range between about 0.5 millimeters to about 1.5 millimeters, and may be desirably about 1.0 millimeters. The length of the operative element 110 may be about 50 millimeters to about 60 millimeters, although it is to be appreciated that the length may vary depending on the particular application. As shown, the operative element 110 may include one or more bends to facilitate accurate placement of the stimulating surface 111. In one embodiment, the conductive material of operative element 110 is made of a stainless steel 304 solid wire, although other known conductive materials may be used.

As previously described, in monopolar operation, a return electrode (or indifferent electrode) 130 or 131, for example, provides an electrical path from the body back to the control device 22 within the housing 112. The return electrode 130 (see FIG. 3A) may be placed on the surface of intact skin (e.g., surface electrodes as used for ECG monitoring during surgical procedures) or it might be needle-like 131 (see FIGS. 3B and 3C), and be placed in the surgical field or penetrate through intact skin. The housing's distal end 118 can incorporate a connector or jack 120 which provides options for return current pathways, such as through a surface electrode 130 or a needle electrode 131, having an associated plug 122. It is to be appreciated that a return electrode and associated lead may be an integral part of the stimulation probe 50, i.e., no plug or connector, as shown in FIG. 3C.

Additionally, the device 50 may desirably incorporate a visual or audio indicator 126 for the surgeon. This visual or audio indicator 126 allows the surgeon to confirm that the stimulator 50 is delivering stimulus current to the tissue it is contacting. Through the use of different tones, colors, different flash rates, etc., the indicator 126 (which can take the form, e.g., of a light emitting diode (LED)) allows the surgeon to confirm that the stimulating tip 111 is in place, the instrument is turned ON, and that stimulus current is flowing. Thus the surgeon has a much greater confidence that the failure to elicit a muscle contraction is because of lack of viable nervous tissue near the tip 111 of the stimulator 50 rather than the failure of the return electrode connection or some other instrumentation problem.

As a representative example, in use the indicator 126 may be configured to illuminate continuously in one color when the stimulation probe 50 is turned on but not in contact with tissue. After contact with tissue is made, the indicator 126 may flash (i.e., blink) to indicate that stimulation is being delivered. If the stimulation has been requested, i.e., the stimulation probe has been turned on, but there is no stimulation being delivered because of a lack of continuity between the operative element 110 and the return electrode 130, or an inadequate connection of the operative element 110 or the return electrode 130 to the patient tissue, the indicator 126 may illuminate in a different color, and may illuminate continuously or may flash.

In one embodiment, as can be best seen in FIGS. 3C and 5, the indicator 126 comprises a ring indicator 128 that provides a visual indication around at least a portion, and desirably all of the circumference of the stimulation probe 50 generally near the flexible nose cone 62. The visual ring indicator 128 may be an element of the gripping portion 60, or it may be an element of the flexible nose cone 62, or the ring indicator may positioned between the gripping portion 60 and the flexible nose cone 62. The ring indicator 128 may also include a reflective element 129 to improve and focus the illumination effect of the light emitting source, e.g., one or more LEDs. The ring indicator 128 and the reflective element may be a single component, or more than one component (as can be seen in FIGS. 5 and 15).

Audio feedback also makes possible the feature of assisting the surgeon with monitoring nerve integrity during surgery. The insulated lead 124 connects to the operative element 110 that, in use, is positioned within the surgical field on a nerve distal to the surgical site. Stimulation of the nerve causes muscle contraction distally. The stimulation control device 22 incorporated within the housing 112 may be programmed to provide an audio tone followed by a stimulation pulse at prescribed intervals. The audio tone reminds the surgeon to observe the distal muscle contraction to confirm upon stimulation that the nerve is functioning and intact.

FIG. 15 shows an exploded view of a representative stimulation probe 50. As can be seen, the stimulation control device 22 is positioned within the housing 112. A battery 34 is electrically coupled to the control device 22. A first housing element 90 and a second housing element 92 partially encapsulate the control device 22. The ring indicator 128 and the reflective element 129 are coupled to the proximal end of the housing 112. The operative element 110 extends through the nose cone 62 and couples to the control device 22. Desirably, the stimulation probe 50 will be constructed in a manner to conform to at least the IPX1 standard for water ingress.

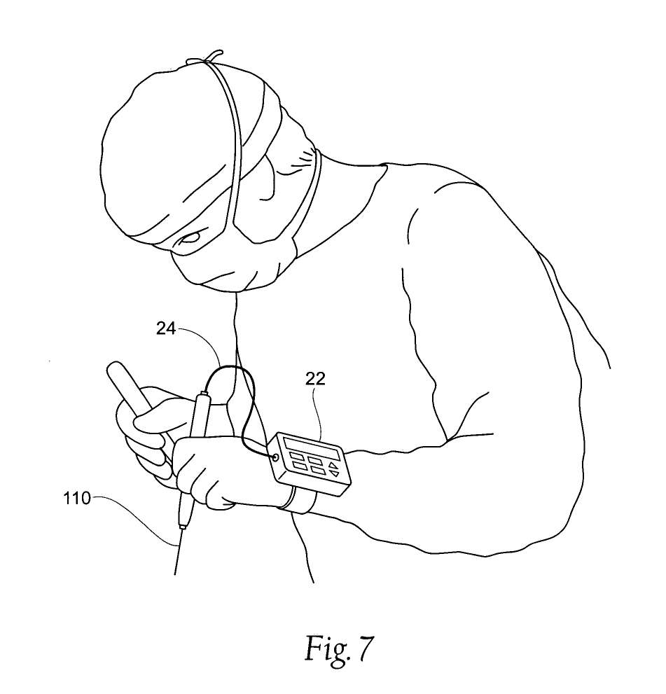

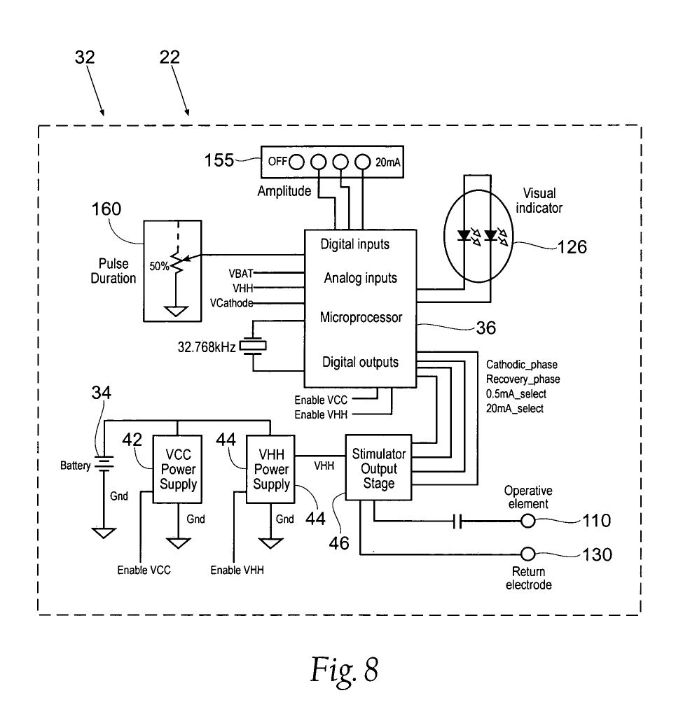

Alternatively, as FIG. 2 shows, the stimulation control device 22 may be housed in a separate case, with its own input/output (I/O) controls 26. In this alternative arrangement, the stimulation control device 22 is sized small enough to be easily removably fastened to a surgeon's arm or wrist during the surgical procedure, or otherwise positioned in close proximity to the surgical location (as shown in FIG. 7), to provide sufficient audio and/or visual feedback to the surgeon. In this arrangement, the separate stimulation control device 22 can be temporarily coupled by a lead to a family of various medical devices for use.

The present invention includes a method of identifying/locating tissue, e.g., a nerve or muscle, in a patient that comprises the steps of providing a hand-held stimulation probe 50, 100 as set forth above, engaging a patient with the first operative element 110 and the second electrode 130, moving the power switch 155 to an activation position causing a stimulation signal 29 to be generated by the stimulation control device 22 and transmitted to the first operative element 110, through the patient's body to the second electrode 130, and back to the stimulation control device 22. The method may also include the step of observing the indicator 126 to confirm the stimulation probe 50, 100 is generating a stimulation signal. The method may also include the step of observing a tissue region to observe tissue movement or a lack thereof.

B. The Stimulation Control Device

As FIG. 8 shows, the stimulation control device 22 includes a circuit 32 that generates electrical stimulation waveforms. A battery 34 desirably provides the power. The control device 22 also desirably includes an on-board, programmable microprocessor 36, which carries embedded code. The code expresses pre-programmed rules or algorithms for generating the desired electrical stimulation waveforms using the stimulus output circuit 46 and for operating the visible or audible indicator 126 based on the controls actuated by the surgeon.

In one form, the size and configuration of the stimulation control device 22 makes for an inexpensive device, which is without manual internal circuit adjustments. It is likely that the stimulation control device 22 of this type will be fabricated using automated circuit board assembly equipment and methods.

C. Incorporation with Surgical Devices

A stimulation control device 22 as just described may be electrically coupled through a lead, or embedded within various devices commonly used in surgical procedures (as previously described for the stimulation probe 50).

1. Cutting Device

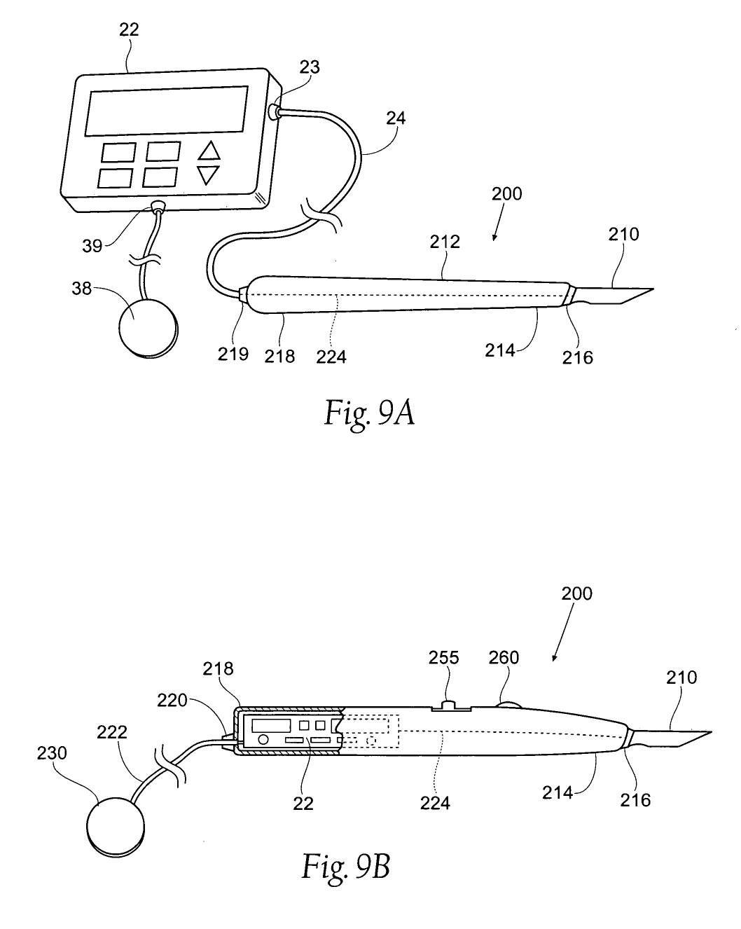

In FIGS. 9A and 9B, a device 200 is shown that incorporates all the features disclosed in the description of the stimulation probe 50, 100, except the device 200 comprises the additional feature of providing an “energized” surgical device or tool. FIG. 9A shows the tool to be a cutting device 200 (e.g., scalpel) removably coupled to a stimulation control device 22.

In the embodiment shown, the cutting device 200 includes a body 212 that carries an insulated lead 224. The insulated lead 224 connects to an operative element, such as electrode 210, positioned at the body proximal end 214 and a plug-in receptacle 219 at the body distal end 118. The lead 224 within the body 212 is insulated from the body 212 using common insulating means (e.g., wire insulation, washers, gaskets, spacers, bushings, and the like).

In this embodiment, the electrode 210 performs the cutting feature (e.g., knife or razor). The electrode 210 performs the cutting feature in electrical conductive contact with at least one muscle, or at least one nerve, or at least one muscle and nerve. The cutting device 200 desirably includes a plug-in receptacle 216 for the electrode 210, allowing for use of a variety of cutting electrode shapes and types (e.g., knife, razor, pointed, blunt, curved), depending on the specific surgical procedure being performed. In this configuration, the lead 224 electrically connects the electrode 210 to the stimulation control device 22 through plug-in receptacle 219 and lead 24.

In one embodiment, the cutting device 200 is mono-polar and is equipped with a single electrode 210 at the body proximal end 214. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the cutting device 200 may be bipolar, which precludes the use of the return electrode 38.

In the embodiment shown in FIG. 9B, the cutting device 200 accommodates within the body 212 the electrical circuitry of the stimulation control device 22. In this arrangement, the cutting device 200 may have at least two operational slide controls, 255 and 260. Power switch 255 serves a dual purpose of turning the stimulation signal to the cutting device 200 on and off, and also is stepped to control the stimulation signal amplitude selection from a predefined range (e.g., 0.5, 2.0, and 20 mA). The pulse control switch 260 allows for adjustment of the stimulation signal pulse width from a predefined range (e.g., zero through 200 microseconds).

At the body distal end 218, a second plug-in receptacle 220 may be positioned for receipt of a second lead 222. Lead 222 connects to electrode 230 which functions as a return path for the stimulation signal when the cutting device 200 is operated in a mono-polar mode.

Additionally, the device 200 may incorporate a visual or audio indicator for the surgeon, as previously described.

The present invention includes a method of identifying/locating tissue, e.g., a nerve or muscle, in a patient that comprises the steps of providing cutting device 200 as set forth above, engaging a patient with the first electrode 210 and the second electrode 230, moving the power switch 255 to an activation position causing a stimulation signal 29 to be generated by the stimulation control device 22 and transmitted to the first electrode 210, through the patient's body to the second electrode 230, and back to the stimulation control device 22. The method may also include the step of observing the indicator 126 to confirm the cutting device 200 is generating a stimulation signal. The method may also include the step of observing a tissue region to observe tissue movement or a lack thereof.

2. Drilling Device

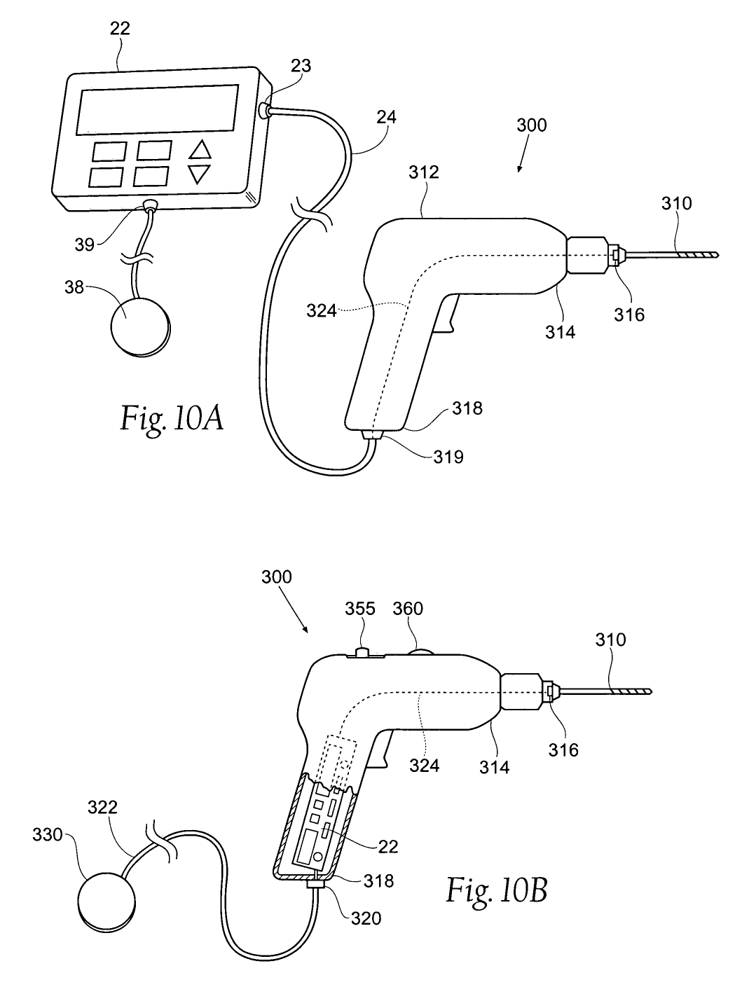

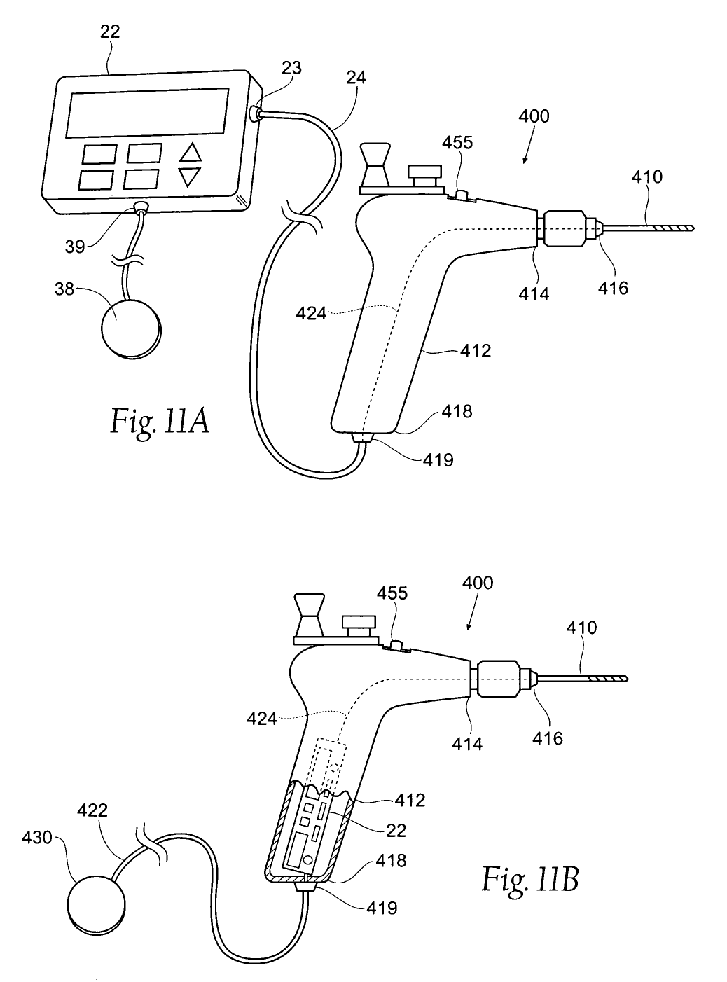

In FIGS. 10A and 10B, a device 300 is shown that incorporates all the features disclosed in the description of the stimulation probe 50, 100, except the device 300 comprises the additional feature of providing an “energized” surgical device or tool, which comprises a drilling device 300. In FIG. 10A is drilling device 300 is removably coupled to a stimulation control device 22.

In the embodiment shown, the drilling device 300 includes a body 312 that carries an insulated lead 324. The insulated lead 324 connects to an operative element, such as electrode 310, positioned at the body proximal end 314 and a plug-in receptacle 319 at the body distal end 318. The lead 324 within the body 312 is insulated from the body 312 using common insulating means (e.g., wire insulation, washers, gaskets, spacers, bushings, and the like).

In this embodiment, the electrode 310 performs the drilling feature. The electrode 310 may also perform a screwing feature as well. The electrode 310 performs the drilling feature in electrical conductive contact with a hard structure (e.g., bone).

The drilling device 300 desirably includes a plug-in receptacle or chuck 316 for the electrode 310, allowing for use of a variety of drilling and screwing electrode shapes and sizes (e.g., ¼ and ⅜ inch drill bits, Phillips and flat slot screw drivers), depending on the specific surgical procedure being performed. In this configuration, the lead 324 electrically connects the electrode 310 to the stimulation control device 22 through plug-in receptacle 319 and lead 324.

In one embodiment, the drilling device 300 is mono-polar and is equipped with a single electrode 310 at the body proximal end 314. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the drilling device 300 may be bipolar, which precludes the use of the return electrode 38.

In FIG. 10B, the drilling device 300 is shown to accommodate within the body 312 the electrical circuitry of the stimulation control device 22. The drilling device 300 may have at least two operational slide controls, 355 and 360. Power switch 355 serves a dual purpose of turning the stimulation signal to the drilling device 300 on and off, and also is also stepped to control the stimulation signal amplitude selection from a predefined range (e.g., 0.5, 2.0, and 20 mA). The pulse control switch 360 allows for adjustment of the stimulation signal pulse width from a predefined range (e.g., zero through 200 microseconds). At the body distal end 318, a second plug-in receptacle 320 may be positioned for receipt of a second lead 322. Lead 322 connects to electrode 330 which functions as a return path for the stimulation signal when the drilling device 300 is operated in a mono-polar mode.

Additionally, the device 300 may incorporate a visual or audio indicator for the surgeon, as previously described.

The present invention includes a method of identifying/locating tissue, e.g., a nerve or muscle, in a patient that comprises the steps of providing a drilling device 300 as set forth above, engaging a patient with the first electrode 310 and the second electrode 330, moving the power switch 355 to an activation position causing a stimulation signal 29 to be generated by the stimulation control device 22 and transmitted to the first electrode 310, through the patient's body to the second electrode 330, and back to the stimulation control device 22. The method may also include the step of observing the indicator 126 to confirm the drilling device 400 is generating a stimulation signal. The method may also include the step of observing a tissue region to observe tissue movement or a lack thereof.

3. Pilot Auger

An additional aspect of the invention provides systems and methods for controlling operation of a family of stimulating devices comprising a stimulation control device electrically coupled to a pilot auger for hard surface rotary probing.

This embodiment incorporates all the features disclosed in the description of the stimulation probe 50, 100, except this embodiment comprises the additional feature of providing an “energized” surgical device or tool. FIG. 11A shows a pilot auger device 400 removably coupled to a stimulation control device 22. In the embodiment shown, the pilot auger device 400 includes a body 412 that carries an insulated lead 424. The insulated lead 424 connects to an operative element, such as an electrode 410, positioned at the body proximal end 414 and a plug-in receptacle 419 at the body distal end 418. The lead 424 within the body 412 is insulated from the body 412 using common insulating means (e.g., wire insulation, washers, gaskets, spacers, bushings, and the like). In this embodiment, the electrode 410 performs the pilot augering feature. The electrode 410 performs the pilot augering feature in electrical conductive contact with a hard structure (e.g., bone).

The pilot auger device 400 desirably includes a plug-in receptacle or chuck 416 for the electrode 410, allowing for use of a variety of pilot augering electrode shapes and sizes (e.g., 1/32, 1/16, and ⅛ inch), depending on the specific surgical procedure being performed. In this configuration, the lead 24 electrically connects the electrode 410 to the stimulation control device 22 through plug-in receptacle 419 and lead 24.

In one embodiment, the pilot auger device 400 is mono-polar and is equipped with a single electrode 410 at the body proximal end 414. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the pilot auger device 400 may be bipolar, which precludes the use of the return electrode 38.

As FIG. 11B shows. the pilot auger device 400 may accommodate within the body 412 the electrical circuitry of the stimulation control device 22. At the body distal end 418, a second plug-in receptacle 420 may be positioned for receipt of a second lead 422. Lead 422 connects to electrode 430 which functions as a return path for the stimulation signal when the pilot auger device 400 is operated in a mono-polar mode.

The pilot auger device 400 includes a power switch 455. When moved to an activation position, a stimulation signal is generated by the stimulation control device 22. Additionally, the device 400 may incorporate a visual or audio indicator for the surgeon, as previously described.

The present invention includes a method of identifying/locating tissue, e.g., a nerve or muscle, in a patient that comprises the steps of providing a pilot auger device 400 as set forth above, engaging a patient with the first electrode 410 and the second electrode 430, moving the power switch 455 to an activation position causing a stimulation signal to be generated by the stimulation control device 22 and transmitted to the first electrode 410, through the patient's body to the second electrode 430, and back to the stimulation control device 22. The method may also include the step of observing the indicator 126 to confirm the pilot auger device 400 is generating a stimulation signal. The method may also include the step of observing a tissue region to observe tissue movement or a lack thereof.

D. Incorporation with Fixation Devices

An additional aspect of the invention provides systems and methods for controlling operation of a family of stimulating devices comprising a stimulation control device electrically coupled to a fixation device or a wrench or screwdriver for placing the fixation device. A fixation device (e.g., orthopedic hardware, pedicle screws) is commonly used during spinal stabilization procedures (fusion), and internal bone fixation procedures.

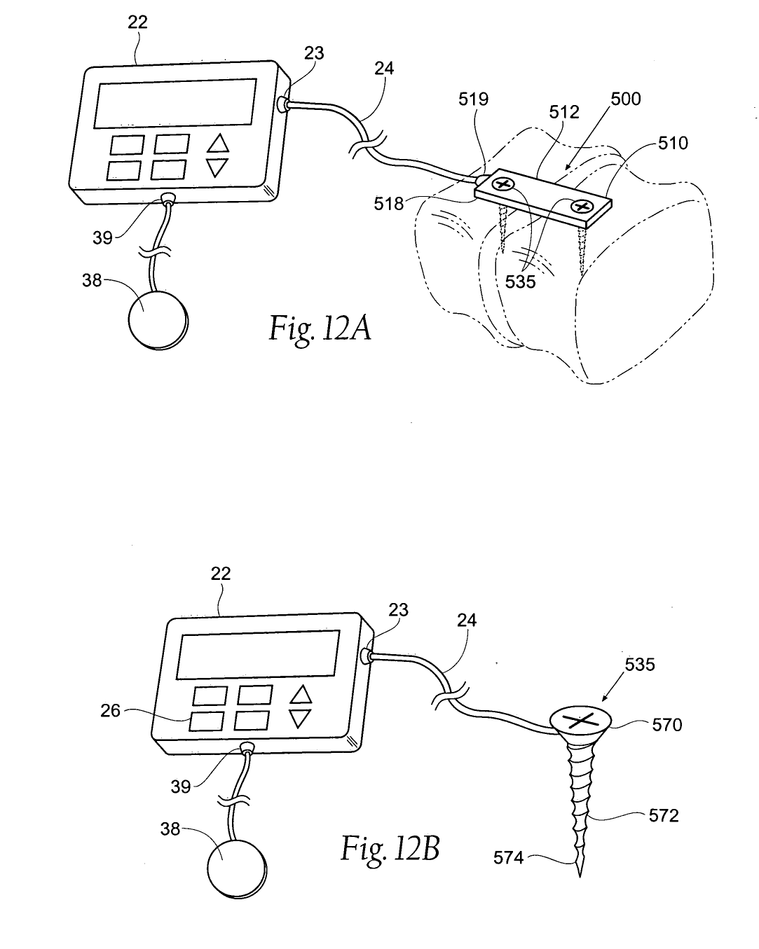

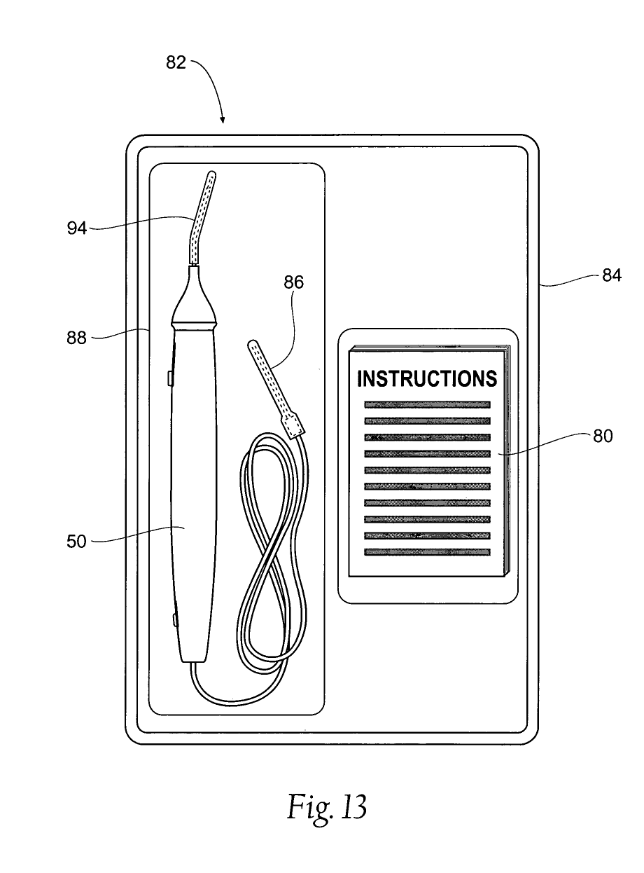

This embodiment incorporates all the features disclosed in the description of the stimulation probe 50, 100, except this embodiment comprises the additional feature of providing an “energized” fixation device or tool. FIG. 12A shows a fixation device 500 removably coupled to a stimulation control device 22. In the embodiment shown, the fixation device 500 includes a rectangularly shaped body 512 that also serves as an operative element, such as electrode 510. The fixation device 500 may take on an unlimited number of shapes as necessary for the particular procedure taking place. Pedicle screws 535 may be used to secure the fixation device to the bony structure. The electrode 510 performs the fixation feature in electrical conductive contact with a hard structure (e.g., bone).

The fixation device 500 or wrench or screwdriver for placing the fixation device desirably includes a plug-in receptacle 519. The fixation device 500 may take on an unlimited variety of shapes and sizes depending on the specific surgical procedure being performed. In this configuration, the lead 24 electrically connects the electrode 510 to the stimulation control device 22 through plug-in receptacle 519.

In one embodiment, the fixation device 500 is mono-polar and is equipped with the single electrode 510. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the fixation device 500 may be bipolar, which precludes the use of the return electrode 38.

In yet an additional alternative embodiment (see FIG. 12B), the fixation device may be a pedicle screw 535. The pedicle screw 535 is removably coupled to a stimulation control device 22. In the embodiment shown, the pedicle screw 535 includes a head 570 and a shaft 572, which both serve as an operative element, such as electrode 574. The electrode 574 performs the fixation feature in electrical conductive contact with a hard structure (e.g., bone), as the pedicle screw 535 is being positioned within a bony structure. The lead 24 electrically connects the electrode 574 to the stimulation control device 22, through a break-away connection or other similar electrical connective means. The fixation device 535 may take on an unlimited variety of shapes and sizes depending on the specific surgical procedure being performed.

In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. In an alternative embodiment, the fixation device 500 may be bipolar, which precludes the use of the return electrode 38.

The present invention includes a method of identifying/locating tissue, e.g., a nerve or muscle, in a patient that comprises the steps of providing a fixation device 500 as set forth above, engaging a patient with the first electrode 510 and the second electrode 38, turning power on to the stimulation control device 22 through the I/O controls 26, causing a stimulation signal 29 to be generated by the stimulation control device 22 and transmitted to the first electrode 510, through the patient's body to the second electrode 38, and back to the stimulation control device 22. The method may also include the step of observing the indicator 126 to confirm the fixation device 500 is generating a stimulation signal. The method may also include the step of observing a tissue region to observe tissue movement or a lack thereof.

### IV. Technical Features

The stimulation control device 22, either alone or when incorporated into a stimulation probe or surgical device, can incorporate various technical features to enhance its universality.

A. Small Size

According to one desirable technical feature, the stimulation control device 22 can be sized small enough to be held and used by one hand during surgical procedures, or to be installed within a stimulation probe or surgical device. The angle of the stimulating tip facilitates access to deep as well as superficial structures without the need for a large incision. Visual and/or audible indication incorporated in the housing provides reliable feedback or status to the surgeon as to the request and delivery of stimulus current.

According to an alternative desirable technical feature, the stimulation control device 22 may also be sized small enough to be easily removably fastened to a surgeon's arm or wrist during the surgical procedure, or positioned in close proximity to the surgical location (as shown in FIG. 7), to provide sufficient audio and/or visual feedback to the surgeon.

B. Power Source

According to one desirable technical feature, power is provided by one or more primary batteries 34 for single use positioned inside the housing and coupled to the control device 22. A representative battery 34 may include a size “N” alkaline battery. In one embodiment, two size “N” alkaline batteries in series are included to provide a 3 volt power source. This configuration is sized and configured to provide an operating life of at least seven hours of operation—either continuous or intermittent stimulation.

C. The Microprocessor/Microcontroller

According to one desirable technical feature, the stimulation control device 22 desirably uses a standard, commercially available micro-power, flash programmable microcontroller 36. The microcontroller 36 reads the controls operated by the surgeon, controls the timing of the stimulus pulses, and controls the feedback to the user about the status of the instrument (e.g., an LED with 1, 2, or more colors that can be on, off, or flashing).

The microcontroller operates at a low voltage and low power. The microcontroller send low voltage pulses to the stimulus output stage 46 that converts these low voltage signals into the higher voltage, controlled voltage, or controlled current, stimulus pulses that are applied to the electrode circuit. This stimulus output stage 46 usually involves the use of a series capacitor to prevent the presence of DC current flow in the electrode circuit in normal operation or in the event of an electronic component failure.

### V. Representative Use of a Stimulation Probe

The stimulation probe 50, 100, as described, make possible the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Instructions for use 80 are desirably included in a kit 82 along with a stimulation probe 50. The kit 82 can take various forms. In the illustrated embodiment, kit 82 comprises a sterile, wrapped assembly. A representative kit 82 includes an interior tray 84 made, e.g., from die cut cardboard, plastic sheet, or thermo-formed plastic material, which hold the contents. Kit 82 also desirably includes instructions for use 80 for using the contents of the kit to carry out a desired therapeutic and/or diagnostic objectives.

The instructions 80 guide the user through the steps of unpacking the stimulation probe 50, positioning the electrodes, and disposing of the single use disposable stimulator 50. Representative instructions may include, but are not limited to:
Remove the stimulation probe 50 from sterile package 88.
Remove cover 94 (e.g., a silicone cover) from the operative element 110.
Remove protective cover 86 from the return electrode 131.
Position the return electrode 131 in contact with the patient such that:
1. The return electrode is desirably positioned in an area remote from the area to be stimulated.
2. The return electrode is desirably not positioned across the body from the side being stimulated.
3. The return electrode is desirably not in muscle tissue.
Turn the stimulation probe 500N by moving the power switch 155 from OFF to the 0.5 mA setting (or greater).
The stimulation probe 50 desirably is turned ON before the operative element 110 makes contact with tissue.
The indicator 126 will be illuminated yellow (for example) continuously if the stimulation probe 50 is ON, but not in contact with tissue.
Contact tissue with the operative element 110.
Adjust the pulse control 160 gradually to increase the level of stimulation. The indicator 126 will flash yellow indicating that stimulation is being delivered.
A flashing red (for example) indicator 126 means that stimulation has been requested, but no stimulation is being delivered because of inadequate connection of the operative element 110 or the return electrode 131 to the patient tissue. Check the return electrode contact and position, and check the operative element 110 contact and position.
Placing the power switch 155 to the off/standby position will stop stimulation and the visual indictor 126 will be illuminated yellow continuously.
Placing the pulse control 160 at the minimum position will stop stimulation and the visual indictor 126 will be illuminated yellow continuously.
A low/depleted battery 34 will cause the stimulation probe 50 to automatically turn OFF and the visual indicator 126 will not be illuminated. No further use of the stimulator 50 will be possible.
At end of use, move the power switch 155 to the off/standby position and move the pulse control 160 to the minimum position.
Cut off and dispose of the return electrode 131 in an appropriate sharps/biohazard container.
Dispose of the stimulation probe 50 per hospital or facility guidelines.

Methods according to the present invention provide the ability to intraoperatively stimulate contraction of a muscle to predict motor capability of a body part after surgery, and to simulate eventual, potential postoperative function of the body part. For instance, tendon transfer procedures are recognized as appropriate treatment in circumstances where nerve damage is present, such as nerve damage caused by a fall, by laceration, by neuropathy, or otherwise. In a tendon transfer procedure, a tendon associated with a working muscle is removed from its natural location and transferred to another location, so as to restore the lost function of the musculotendinous unit that may have been paralyzed by trauma or intrinsic nerve or muscle pathology, which may have caused the loss of function. The working muscle may be selected from a group of muscles including synergistic muscles associated with the non-working muscle. The transferred tendon is sutured in place at a desired tension, and the surgical operation is completed conventionally. However, there has always been a problem in judging the appropriate tension and/or the likely outcome of a tendon transfer procedure intraoperatively. Indeed, such problem was recognized by Podolsky, et al. in U.S. Pat. No. 4,693,606, directed to determination of muscle sarcomere length in vivo.

As indicated by Podolsky, adjustment of muscle length during tendon transfer procedures and tendon repairs is a necessary element in obtaining maximum force and/or desired motor response from the transferred muscle. A muscle that is set at a length different than the optimal muscle length will not be able to develop maximum force and will have a different length of excursion that will affect function and motion. Usually, surgeons rely on their own experience in determining the amount of tension for setting a muscle to its optimal muscle length, and it is not uncommon for the surgeon to overpull the muscle during the tendon transfer.

Adjustment of muscle length during tendon transfers has always presented a problem for a surgeon, because the force developed by an activated muscle is length-dependent. The objective of the surgeon in any muscle and/or tendon transfer should be to situate the muscle associated with the transferred tendon at its optimal length for the desired motor response. Previously, guidelines for adjusting muscle length generally were based on total muscle excursion and the passive tension felt during surgery. The passive tension felt during surgery depends not only on the length of the muscle and inherent qualities of the individual muscle fibers, but also on the surrounding connective tissue adhesions.

While passive tension is important for the assumption of functional position, it is an inadequate predictor of muscle function. If the muscle is paralyzed or under tourniquet control, extrinsic factors are eliminated, and the muscle exerts passive tension but not active tension. Thus, methods for tendon transfer that depend on passive tension give no information on how the muscle will later function in the patient. Moreover, there are no accurate practical clinical or physiologic guidelines for assuring that optimal muscle length is restored following tendon transfer procedures.

To date, there have been estimated rules of thumb, but no semi-quantitative way for determining proper tensioning of a transferred muscle during a tendon transfer procedure. For instance, Mayer's rule dictates that, in certain circumstances, the origin of the muscle to be transferred should be positioned as close to the insertion as possible, and the tendon is then sutured under no added tension. However, a surgeon often relies on hands-on experience with such procedures to determine the approximate insertion location. Such experience does not necessarily take into account the unique characteristics of the transferred muscle. That is, the transferred muscle and its innervated nerve have properties that are unobservable to the naked eye during the surgical operation. Thus, sometimes, despite being performed by the most experienced surgeon, a tendon transfer is unsuccessful due to improper tensioning or unanticipated effects of the transfer.

Accordingly, improved methods according to the present invention include intraoperative physiologic functional simulation by electrical stimulation during, for example, a tendon transfer procedure. While a tendon transfer procedure is described in detail, the invention will readily apply to any surgical procedure during which electrical stimulation may be used to predict the functional success of a neural response after completion of the procedure. A neural response may range from a muscle twitch to a complete motor response of a body part through a desired range of motion. In one embodiment of a method according to the present invention, a gracilis to bicepstransfer procedure is performed. In a first series of steps, the gracilis to bicepstransfer progresses as is recognized in the art.

After securing or temporary stabilization of a temporary stitch, which couples the transferred tendon to bone, such as by stitching to a periosteum of such bone tissue, or to another muscle, or another tendon, electrical stimulation may be applied to the nerves that innervate the transferred muscle. The stimulation is preferably provided by a handheld electrical stimulator as herein described. The stimulation may be provided having static parameters, such as amplitude and duration, or one or more of the parameters may be altered during the stimulation to achieve a stimulation intensity adequate to create a motor response. While the electrical stimulation is being applied, a motor response is observed. The motor response is a motion of the body part, or extension thereof, to which the transferred tendon is connected. If the motor response is as desired, predicted or expected, the transferred tendon may be secured in place at the tension provided by the temporary stitch. If the motor response is not as desired, the tension of the transferred tendon may be adjusted, or insertion altered, to achieve desired motor response. After adjustment, additional electrical stimulation may be applied to confirm the desired motor response.

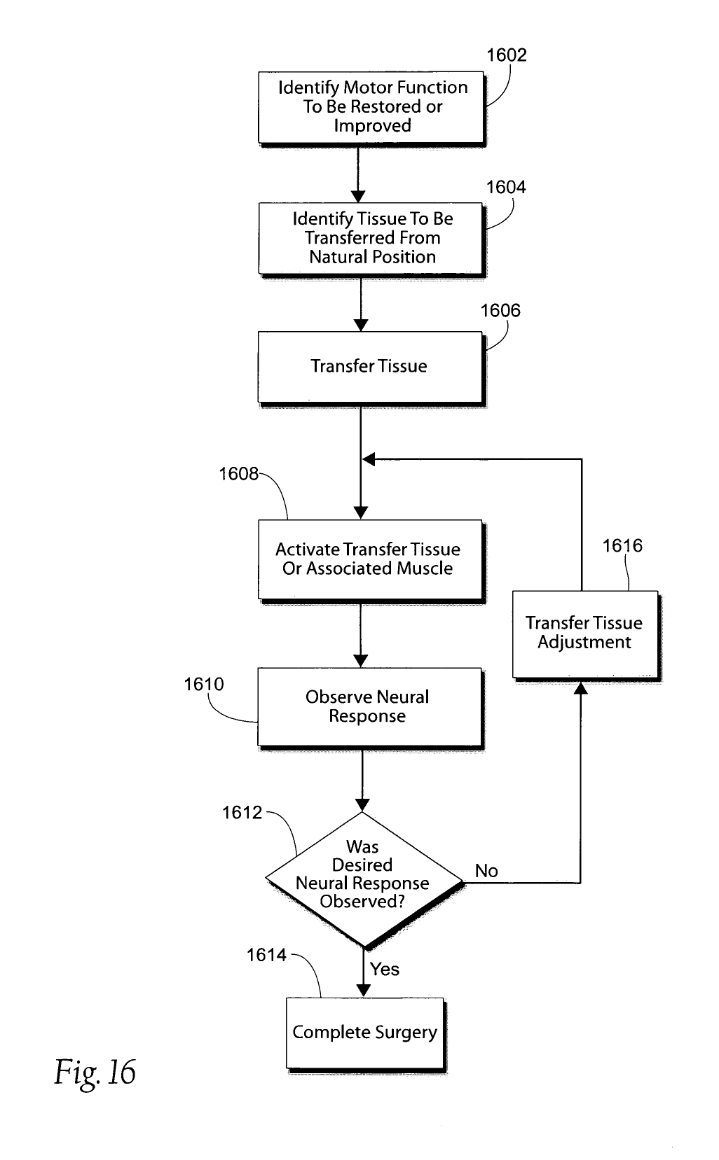

An embodiment of a method according to the present invention may be seen in FIG. 16. In one step 1602, a relocation or restoration of motor function is determined. The motor function may have been damaged as a result of injury or neuropathy, for example. In another step 1604, viable muscle and/or tendon tissue may be identified. Such identified tissue may be referred to as the transfer tissue. The identification step may be simply visual, performed by a surgeon, or it may involve electrical stimulation of the transfer tissue to confirm physiologic function. In another step 1606, the identified transfer tissue may be transferred. For instance, a tendon associated with or comprising the transfer tissue may be identified and incised or severed.

For example, an injury to a radial nerve, caused, for example, by fracture, or by blunt or penetrating trauma, may result in paralysis and an inability to extend (straighten) the wrist and/or fingers. A recommended treatment for such injury may include one or more tendon transfers, whereby normally working muscle-tendon units, which may be expendable without significant functional loss, are detached from their normal anatomical location and transferred to substantially replace the non-functioning muscle-tendon units to imitate their function. In the case of a radial nerve palsy, one of the wrist flexor muscles (commonly the flexor carpi radialis) may be transferred and sewn to finger extensor tendons (the extensor digitorum communis), the pronator teres tendon and associated muscle may be transferred to the wrist extensor (extensor carpi radialis longus) and the Palmaris longus tendon (a vestigal muscle) may be transferred to restore thumb extension (extensor pollicis longus). Thus, muscles that are naturally adapted to rotate the forearm and flex the wrist may be transferred to restore extension of the wrist, fingers and thumb. In yet another step 1608, preferably after the transfer 1606, the transfer tissue or muscle associated therewith, may be activated by sufficient electrical stimulation to, for example, determine whether the transferred tissue has been positioned in an appropriate manner to achieve the desired response. A neural response (which may be a motor response) is observed 1610. A determination 1612 is made as to whether the observed neural response was the desired neural response. If so, the surgery may be completed 1614, such as by more permanently securing the transfer tissue or associated tissue at the last-tested position. If the observed neural response is not a desired neural response, or if no response is observed, a transfer tissue adjustment 1616 may be made, such as by adjusting the length of a transferred muscle or suture position of a transferred tendon, or both. After the tissue adjustment 1616, the transfer tissue may be activated again 1608 to determine if a desired neural response may be generated. The process of tissue transfer adjustment 1616 and transfer tissue activation/response 1608-1612 may be repeated one or more times until a desired response is achieved.

Although an embodiment of a physiologic functional simulation has been described in-connection with a tendon transfer procedure, it is to be understood that the method may be used in other situations, as well. For instance, physiologic functional simulation may be employed as a diagnostic method. In other words, it may be desirable to stimulate nerve fibers that innervate muscle tissue to determine whether a desired neural response is achieved, which may lead to a diagnosis of neuropathy. Accordingly, while a tendon transfer and like procedures involve placing a muscle in an unnatural orientation or configuration, it may be desirable to maintain a muscle in its natural configuration and to simulate physiologic function by electrical stimulation to determine whether certain nerve fibers are viable.

Additionally, another situation in which physiologic functional simulation, without unnatural positioning, may be desirable is during surgery unrelated to a specific target muscle or nerve. For instance, it may be desirable to document a pre-surgery, simulated physiologic functional response prior to a given surgery in response to an applied stimulation, to perform the given surgery, and then to apply stimulation after the surgery to document a post-surgery, simulated physiologic functional response. A comparison between the post-surgery response and the pre-surgery response may be made to determine the effect, if any, of the given surgery upon the physiologic function of the stimulated tissue.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. In a surgical procedure affecting functional change of muscle tissue innervated by working nerve tissue, a method of physiologic functional simulation comprising the steps of:
relocating first tendon tissue to an unnatural position;
in a first application step, applying a first electrical stimulation to first muscle tissue into which the first tendon tissue is naturally inserted; and
in a first observation step, observing a neural response to the first electrical stimulation.

2. A method according to claim 1, wherein the first tendon tissue comprises a first tendon having two ends, wherein a first end is inserted into the first muscle tissue and a second end is coupled to a first bone, the method further comprising the steps of:
incising all or part of the first tendon at or near the first bone to form a tendon free end; and
coupling the tendon free end to a second body part.

3. A method according to claim 2, wherein the second body part is a second bone.

4. A method according to claim 3, wherein the tendon spans a joint between the second bone and a third bone.

5. A method according to claim 2, wherein the relocating step is performed before the first application step.

6. A method according to claim 5, wherein the neural response is a movement of the second body part through a predetermined range of motion.

7. A method according to claim 2, wherein the incising step comprises the step of severing the first tendon.

8. A method according to claim 7, wherein the coupling step comprises the step of stitching the first tendon to one of a second tendon, a second muscle, and a periosteum of the second bone.

9. A method according to claim 1, the method further comprising the steps of:
providing a device comprising:
a housing extending along a housing longitudinal axis between a housing proximal end and a housing distal end;
electrical stimulation generation circuitry at least substantially contained within the housing;
an operative element extending from the housing proximal end, the operative element comprising an electrode operatively coupled to the electrical stimulation generation circuitry; and
a power supply disposed at least substantially within the housing, the power supply being electrically coupled to the electrical stimulation generation circuitry; and
wherein the first application step comprises the step of applying the first electrical stimulation from the electrode to the first muscle tissue.

10. A method according to claim 9, wherein the device is at least substantially sterile.

11. A method according to claim 9, wherein the stimulation circuitry is contained entirely within the housing.

12. A method according to claim 9, wherein the device is capable of delivering, through the electrode sufficient electrical stimulation to activate the first muscle tissue when the electrode is in physical contact therewith.

13. A method according to claim 9, wherein the power supply is contained entirely within the housing.

14. A method according to claim 9, further comprising the step of carrying the device by a single human hand.

15. A method according to claim 14, further including the step of manipulating, with the single human hand, the device to change an electrical stimulation parameter.

16. A method according to claim 9, the device further comprising an electronic visual indicator operatively coupled to the stimulation circuitry.

17. A method according to claim 16, further comprising a step of observing a first visual indication provided by the visual indicator, the first visual indication being indicative of electrical stimulation flowing at least partially through the targeted tissue region.

18. A method according to claim 17, further comprising a step of observing a second visual indication provided by the visual indicator, the second visual indication being indicative of electrical power supplied to the stimulation circuitry by the power supply.

19. A method according to claim 18, wherein the first visual indication is an illumination of a first color and the second visual indication is an illumination of a second color, the second color being different from the first color.

20. A method according to claim 19, wherein the visual indicator is an illumination device that is radially visible from 360 degrees around the longitudinal axis of the handle.

21. A method according to claim 15, wherein the manipulating step occurs during the first application step.

22. A method according to claim 1, wherein the neural response is at least a twitch of the first muscle.

23. A method according to claim 1, wherein the first application step and the first observation step are performed before the relocating step, further comprising, after the relocating step, the steps of:
in a second application step, applying a second electrical stimulation to the first muscle tissue; and
in a second observation step, observing a second neural response to the second electrical stimulation.

24. A method according to claim 1, further comprising the step of, prior to the relocating step, confirming a paralysis of a second muscle, the function of which will be supplemented by the first muscle tissue.

25. A method according to claim 24, wherein the confirming step is performed, in a confirmation step, by applying a second electrical stimulation to the second muscle, the electrical stimulation having an amplitude of about twenty milliamps.

26. A method according to claim 25, wherein the first application step and the first observation step are performed before the relocating step, further comprising, after the relocating step, the steps of:
applying a third electrical stimulation to the first muscle tissue; and
observing a second neural response to the third electrical stimulation.
